(19) United States
(12) Patent Application Publication
(10) Pub. No.: US 2016/0223509 A1
(43) Pub. Date: Aug. 4, 2016

(54) METHOD AND DEVICE FOR ANALYSING SULFATES IN A LIQUID

(71) Applicant: INSTITUT FRANCAIS DE RECHERCHER POUR L'EXPLOITATION DE LA MER-IFREMER, Issy-Ies-Moulineaux (FR)

(72) Inventors: Florian CARADEC, Lennon (FR); Agathe LAËS-HUON, Brest (FR); Sophie CHATAING-PARIAUD, Locmaria-Plouzane (FR); Karenn BUCAS, Lannilis (FR)

(21) Appl. No.: 15/022,453

(22) PCT Filed: Sep. 8, 2014
(86) PCT No.: PCT/FR2014/052214

§ 371 Date: Mar. 16, 2016

(30) Foreign Application Priority Data
Sep. 16, 2013 (FR) 1358912

(51) Int. Cl. G01N 33/18; G01N 21/75

(52) U.S. Cl. CPC G01N 33/182; G01N 21/75; G01N 2201/062; G01N 2201/0826

(57) ABSTRACT

The invention relates to an analysis device (1) comprising: a tight analytical circuit (3) comprising an injection loop (BI), at least one mixing loop (BM), and a turbidimetry analysis means (CD), a liquid being able to continuously circulate through the injection loop, the mixing loop and the analysis means; a means (IL) for injecting said liquid into said injection loop and a means for removing said liquid from a source (S); a means (ISR) for injecting, into said injection loop, a solution for displaying sulfates in said liquid by means of turbidimetry; and a means (ISP) for injecting a rinsing solution, comprising a peristaltic pump (PPB) and a system (VF, VG) of valves placed between the pump (PPB) and said injection loop. The invention is especially applicable in fields of the offshore oil industry.

### TECHNICAL FIELD

The present invention relates to a device for analyzing the amount of sulfates contained in a liquid, and also to the use of the device for analyzing a liquid, especially seawater, especially nanofiltered seawater.

The present invention especially finds applications in the fields of the offshore oil industry, especially the exploitation of deposits, for example oil deposits, and also in industrial processes such as the manufacture of paper and cellulose, the manufacture of edible oils, tanneries, environmental monitoring, water treatment, etc.

In the description below, the references between brackets ([ ]) refer to the list of references presented at the end of the examples.

### BACKGROUND

The oil industry uses various techniques for exploiting deposits, including that of injecting water into the natural reservoirs of these deposits in order to increase the pressure and thus promote the extraction of the oil. The water injected generally originates from the surrounding seawater in the case of offshore exploitations.

This seawater, rich in sulfates, may lead to the formation of precipitates of barium sulfates, strontium sulfates and calcium sulfates, these ions being present in the waters of the natural reservoirs. This phenomenon creates numerous problems with respect to the installations for extracting these deposits, in particular a drop in the production efficiency and also an increase in the maintenance operations of these installations.

In order to prevent problems of this type, the seawater is first desulfated by a nanofiltration process.

The content of sulfates at the outlet of the nanofiltration process is generally monitored in order to verify the sulfate concentration of the injected waters, also referred to as injection waters. The operator or the person responsible for maintenance determines a threshold value, for example a sulfate concentration around 40 mg·L⁻¹ (0.42×10⁻³ mol/L or 0.42 mM), which determines the stopping of the injection of the nanofiltered waters and the beginning of maintenance interventions of the installations. These interventions comprise, for example, the rinsing of the installations, any part of the device sensitive to clogging, etc.

They require the stopping of the extraction and significant human, equipment and reagent means. It is therefore essential to have an effective and economical means of analyzing these sulfates that makes it possible to optimize these interventions and to minimize the costs thereof.

In the case of an application in the offshore oil industry, many constraints must be considered. Firstly, it is necessary to have an on-line measurement of the sulfates. Indeed, a manual sampling then an analysis is not desirable in this case, since this sampling leads to supplementary maintenance operations and costs, involving a need for personnel and a possible contamination of the liquid to be analyzed.

Moreover, in order to limit the maintenance operations linked to the analysis device itself, it is necessary to have an analysis device that is economical in terms of reagents and which has sufficient energy autonomy not to itself be subjected to time-consuming and expensive maintenance operations.

Furthermore, there are currently no automated devices that are practical, reliable, suitable and of appropriate size to be able to be positioned easily within deposit-extracting installations.

The sulfate analyzers of the prior art unfortunately do not fulfil all of these conditions, since these analyzers of the prior art are not qualified for measurement in a seawater matrix. For some, a pretreatment by cation-exchange resin is even necessary in order to prevent the interferences with other ions such as calcium ions or else the formation of precipitates.

Moreover, the measurement accuracies, or measurement ranges, of the apparatus of the prior art are relatively limited. Some have bottom limits of greater than 20 mg·L⁻¹ (0.21×10⁻³ mol/L or 0.21 mM) or are not specified for low values of sulfates, for example from 0 to 20 mg·L⁻¹ (0 to 0.21×10⁻³ mol/L or 0 to 0.21 mM) or even 0 to 50 mg·L⁻¹ (0 to 0.52×10⁻³ mol/L, 0 to 0.52 mM) as bottom limits.

Known from among the methods used for the analysis of concentrations of sulfates contained in a liquid is, for example, the method described in the publication by Van Staden, 1987, On-line sulphate monitoring by reversed flow injection analysis and alternating reagent injection, Fresenius Z Anal Chem 326: 754-756 [1]. Nevertheless, the method described in this document is not qualified for the analysis of seawater. Furthermore, the detection limit described in the document is 30 mg·L⁻¹ (0.31×10⁻³ mol/L or 0.31 mM); this value is too high for the analysis of a nanofiltered seawater.

There is therefore a real need for a device and a process for analyzing the amount of sulfate contained in a liquid that overcome these failings, drawbacks and obstacles of the prior art.

### SUMMARY OF THE INVENTION

The present invention makes it possible precisely to solve all of the technical problems and drawbacks of the prior art by providing, in particular, a device (appended FIG. 1) (1) for analyzing sulfates in a liquid (L), said device comprising the following components:
a) a leaktight analytical circuit (3) comprising an injection loop (BI) of defined volume connected to at least one mixing loop (BM), and a turbidimetric analysis means (CD), it being possible for the injection loop, the mixing loop and the analysis means to be passed through continuously and successively by a liquid, the injection loop and the mixing loop being separable by a valve (VH);
b) an injection means (IL) for injecting said liquid into said injection loop, said injection means comprising a peristaltic pump (PPA), a system of valves (VF, VG) placed between the pump (PPA) and said injection loop, and a means for sampling said liquid from a source (S);
c) an injection means (ISR) for injecting, into said injection loop, a solution for detecting sulfates in said liquid by turbidimetric analysis; said injection means (ISR) comprising a peristaltic pump (PPC) for injecting said solution for detecting sulfates and a valve (VG) placed between said pump (PPC) and said injection loop; and
d) an injection means (ISP) for injecting a solution for rinsing said analytical circuit, said injection means (ISP) comprising a peristaltic pump (PPB) for injecting said rinsing solution and a system of valves (VF, VG) placed between the pump (PPB) and said injection loop.

The device of the present invention has been named by the inventors as “CHEMINI Sulfates”, for “CHemical MINIaturized analyzer Sulfates”. It is a miniaturized device or instrument developed for the on-line measurement of sulfates in a liquid. It is a chemical analyzer that has, in particular, the advantage of being able to be used in situ, and it is based on a reversed flow injection (or “rFIA” for “reversed flow injection analysis”) and a turbidimetric detection. The objective of CHEMINI Sulfates is to measure the concentration of sulfates in a liquid, especially at the outlet of nanofiltration modules in order to monitor the efficiency of the latter and to trigger, if necessary, the interventions useful for the maintenance thereof. It advantageously makes it possible to meet all of the specific requirements for the analysis of sulfates for the oil industry.

The term “liquid” is understood to mean any liquid for which there is an interest in quantifying the amount of sulfates contained in said liquid. It may for example be an effluent, for example seawater that is optionally nanofiltered. For example, it may be nanofiltered seawater used in the case of exploitation of natural deposits, for example oil, for example during offshore oil extraction, where the process of the present invention is particularly advantageous. It may also be on-line measurements of sulfates in industrial process waters, for example for the manufacture of paper and/or cellulose, for the manufacture of edible oils, in tannery processes, for carrying out environmental monitoring, in the treatment of potable or non-potable waters, etc.

In addition, the device of the present invention is highly advantageous from the ecological and economic viewpoint, since it makes it possible to economize and optimize the human, equipment, maintenance operation and reagent means used. It also has an industrial interest, since it enables the production to be increased due to the limitation of the maintenance operations. It also has an interest in the field of laboratory analysis when continuous flow analyses are necessary.

In the device of the present invention, the analytical circuit is leaktight. The term “leaktight” is understood to mean impermeable to liquids and gases, including under internal or external pressure. The leaktightness of the device may be ensured by an appropriate choice of the materials constituting it and by the use of appropriate leaktight seals or welds and/or by containment of part or all of the device in a leaktight container and/or in a liquid under equal pressure, for example oil, for example dielectric oil.

The leaktightness may also be ensured by the positioning or containment of part or all of the device in a leaktight casing, which may be plastic, glass or metal, for example made of titanium. The materials used to form the device of the invention will be easily selected by a person skilled in the art so that this leaktightness is ensured. The examples below present examples of containment of the device of the present invention.

According to the invention, the injection loop has a defined volume that makes it possible to inject a defined amount of solution for detecting sulfates present in the liquid to be analyzed by turbidimetric analysis. According to the invention, the injection loop may have for example an internal diameter of from 0.20 to 1 mm, for example from 0.60 to 1 mm, for example 0.80 millimeter. The length of the injection loop may be for example from 3 to 10 cm, for example from 4 to 8 cm, for example 6 centimeters.

The injection loop may be of any shape suitable for the implementation of the invention, for example serpentine or spiral-shaped, preferably wound in a spiral, which makes it possible to obtain a compact device.

In the device of the present invention, the injection loop is connected to at least one mixing loop. This connection is preferably leaktight and may be separated by a valve (VH), it being possible for the liquid to be analyzed to pass continuously through the injection loop then the mixing loop.

According to the invention, the mixing loop may have for example an internal diameter identical to or different from that of the injection loop, for example of from 0.20 to 1 mm, for example from 0.60 to 1 mm, for example 0.80 millimeter. The length of the mixing loop may be for example from 1 to 3 m, for example from 1.5 to 2.5 m, for example 2 m.

The mixing loop may be of any shape suitable for the implementation of the invention, for example serpentine or spiral-shaped, preferably wound in a spiral, which makes it possible to obtain a compact device. According to the invention, the device may comprise one or more mixing loops, one after the other, for example two, three or four mixing loops, preferably two mixing loops, which follow one another and which may be passed through continuously by the liquid to be analyzed and by the solution for detecting sulfates for the mixing thereof.

The injection loop and the mixing loop may comprise an identical or different material, preferably an identical material, preferably a material that does not interact with the liquid and the solution for detecting sulfates, and that may also, depending on the usage conditions, have to withstand internal and/or external pressures greater than atmospheric pressure. It may be for example a metal selected from the group comprising stainless steel or a PEEK-type plastic. Advantageously, the metal may be covered on its inner surface by Teflon (registered trademark) or any other material known to a person skilled in the art and that is compatible with the liquid to be analyzed, the sulfates and the reagents used.

The mixing loop enables a mixing of the liquid to be analyzed and of the solution for detecting sulfates optionally present in said liquid, before this mixture passes into the turbidimetric analysis means.

The turbidimetric analysis means makes it possible to detect the sulfates optionally present in the liquid to be analyzed, and to quantify them where appropriate, owing to the interaction of said sulfates with the solution for detecting the latter. This analysis means may be composed of a detection cell (DC), an electronic detection board (CE), LEDs or micro-LEDs, and a photodiode. This device advantageously has 3 LEDs having wavelengths of 520 nm (±30 nm) which is the wavelength for detecting barium sulfate, 650 nm (±20 nm) and 810 nm (±35 nm).

The detection part of the analyzer may be composed, for example, of a measuring cell, which may for example have a length of from 2 to 4 cm, and for example an internal diameter of from 0.5 to 1.5 mm, for example 1 mm, manufactured by HELLMA (Germany). It may comprise an electronic detection board. The electronic board for the detection is for example a board capable of receiving three LEDs and a photodiode. The light emitted by the LEDs is transferred to the measuring cell then is sent back to the photodiode-type detector by optical fibers, for example PMMA optical fibers, with for example an internal diameter of 1 mm.

The barium sulfate precipitate attenuates the incident light of the detection cell. The residual light leaving the detection cell may then be captured by a photodiode.

The detection cell is passed through by the hydraulic circuit. The tubing may be connected to the cell with the aid of connector technology for example of Minstac (registered trademark) (Lee Company, USA) type, the tubing at the outlet may for example be connected to a container for receiving the analyzed liquid (not represented).

The analysis means thus makes it possible to detect, by turbidimetric analysis, a concentration of sulfates in said liquid analyzed by formation of the precipitate, for example BaSO₄, in the mixing loop.

In the present invention, the term “valve” denotes a means used for stopping the liquid or the solution in the device of the present invention. According to the invention, the valves may be any valve known to a person skilled in the art and suitable for the device of the present invention. For example, they may be electrovalves. Known from among the valves used are for example those described in the publication by Vuillemin et al, 2009, CHEMINI: A new in situ CHEmical MINIaturized analyzer, Elsevier, Deep-Sea Research I 56(2009) 1391-1399 [2]. According to the invention, the valves may be solenoid valves, for example three-way solenoid valves (VA to VH).

The “peristaltic pumps” make it possible to inject the liquid and the detecting and rinsing solutions into the device of the present invention. Known from among the pumps used are those described in the publication by Vuillemin et al, 2009 [2]. The peristaltic pumps may be, for example, single-channel peristaltic pumps (PPA, PPB and PPC).

Actuators may be used for the circulation of the various fluids in the device: detecting solution, rinsing solution, standards and liquid to be analyzed.

According to the invention, the source (S) may comprise at least one line (V), for example (V1), (V2), (V3), (V4), (V5), (V6), for sampling the circulating or moving liquid, said line comprising at least two valves (VA, VB, VC, VD, VE) placed between said moving liquid and the peristaltic pump (PPA).

The expression “sampling line (V)” is understood to be a means that makes it possible to connect the device of the present invention to a duct in which the liquid to be analyzed circulates, for example to a seawater nanofiltration module or a circuit for injecting nanofiltered seawater into natural reservoirs, for example oil reservoirs, for example offshore, the seawater constituting said moving liquid (L). Any connection means known to a person skilled in the art for sampling a liquid in a first circuit and conveying it to an analysis circuit may be used.

The nanofiltration module for which it is desired to verify the sulfate filtration quality may be, for example, a desulfation treatment device comprising membrane modules and the monitoring of the sulfate content of which may be carried out on the outlet collector of all of the modules.

According to the invention, the device may comprise a reservoir of solution for detecting sulfates from which the peristaltic pump (PPC) withdraws said solution in order to inject it into the analytical circuit. It may be a flexible plastic transfer pouch made of polyethylene or equivalent or bottling by means of glass bottles.

According to the invention, the device may comprise a reservoir of rinsing solution from which the peristaltic pump (PPB) withdraws said solution in order to inject it into the analytical circuit for the rinsing thereof. It may be a transfer pouch.

The connections between tubes and valves are made by means of MINSTAC (trademark) (Lee Co.) connectors and Luer (trademark) (Fisher) connectors.

According to the invention, the device of the present invention may be automated, for example by means for programming and controlling the continuous reversed flow injection, into said injection loop, of the solution for detecting sulfates in said liquid, said solution possibly comprising for example a stoichiometric amount of BaCl₂ or of an equivalent compound known to a person skilled in the art that makes it possible to detect a maximum concentration of sulfates that is acceptable in said liquid.

According to the invention, these control means are connected for example to the valves (VA, VB, VC, VD, VE, VF, VG and VH), to the peristaltic pumps (PPA, PPB and PPC) and to the analysis means and make it possible to automate the device of the present invention, for example for an automated control of the content of sulfates at the outlet of seawater nanofiltration modules of the offshore oil industry or any other installation requiring a control of the sulfate concentration in a liquid effluent.

This control may be carried out for example by means of a board laid out and designed on the basis of an ATMEL Atmega (8 MHz, 3.3 V) microcontroller. This board preferably has the following features: 64 kb of RAM, 512 kb of Flash data, 128 kb available for the code, SPI and 120 bus, 3 timers, 8 ADC channels, 2 UARTs, 15 I/O ports and dimensions of 55×55 mm. This board makes it possible to manage each component of the device of the invention in detail and also the optical module. The board based on a microcontroller enables the control of the optical module, of the components of the apparatus (electrovalves, pumps) and also the communication with the HMI of a computer.

The device of the present invention may additionally comprise Human-Machine Interface (HMI) operating software, for example developed in Visual Basic or any other system that makes it possible to control the various components of the apparatus and to export the data obtained, for example to calculation software of Excel type. The features of the HMI operating software and CHEMINI embedded software have been filed at L'Agence pour la Protection des Programmes [The Agency for the Protection of Programs] under the reference: IDDN.FR.001.430027.000.R.P.2009.000.30625 (certificate of filing dated Oct. 21, 2009).

Thus, according to the invention, the device may be programmed and controlled by means of a Human-Machine Interface (HMI). According to the invention, this control may for example be such that it may be executed on three levels: a low-level mode, a remote-control mode and an autonomous endurance mode. In low-level mode, the user has access to each component of the device of the invention and each parameter manually. He can modify the rotational speed of a peristaltic pump in particular or optionally switch each valve. This mode may be used for primary development and maintenance operations. In the remote-control mode of the analyzer, the user may launch a pre-recorded program for the implementation of the device of the present invention. This mode may be used for acquisitions of a measurement over the short term or during a relatively simple access to the analyzer. Finally, the autonomous operating mode makes it possible to launch a selection of pre-recorded programs, also referred to as cycles. This function is useful for acquiring long series of measurements or for autonomous operations at a set time over several days for example. The data may be stored in an internal memory of the device of the present invention and may be extracted using the operating software of the analyzer (HMI). The raw data may also be processed by the software in order to obtain the value of the light intensity absorbed by the particles (turbidimetric analysis) and to minimize the effect of the noise of the measurements. The raw and processed data may then be transferred from the HMI software to EXCEL calculation software in order to process the data in greater depth.

Indeed, the device may comprise a memory that makes it possible to memorize successive analyses. For example, it may be an AT45DB321 SPI Flash-type memory, which may for example have a capacity of 32 megabits or more.

According to the invention, the device may for example in its entirety, that is to say its hydraulic module comprising the injection circuit and the mixing circuit, its optical module, comprising the analysis means, operate at 12 V.

According to the invention, the device may communicate with the HMI software for example via an RS-232 serial link.

According to the invention, the device may be contained within at least one leaktight chamber for underwater immersion.

For example, it may be a single leaktight chamber. This version is especially suitable for coastal use of the device of the invention. For example, it may be a chamber that can be immersed to 10 meters or more depending on the materials used and the leaktightness thereof. This chamber may be made of stainless steel having a thickness of 1.5 mm or made of polymethyl methacrylate (PMMA) having a thickness of 10 mm. The dimensions of this chamber are suitable for receiving the device of the invention, for example from 20 to 25 cm high, for example from 12 to 16 cm long and from 10 to 14 cm wide for a device defined in order to be inserted therein.

According to the invention, the device may be composed of two separate parts or blocks, one comprising the injection and mixing loops or hydraulic module, and the other comprising the turbidimetric analysis means or optical module and electronics. This version may be useful as “deep sea” version (DVGF), for example that can be immersed down to 6000 meters. In this case, the hydraulic module is preferably at equal pressure, for example in dielectric oil, for example Fluorinert, FC77, 3M (trademark) (3M, USA), with containment in a casing, of which the upper cover may be made of transparent PMMA, the lower cover may be made of polyvinyl chloride (PVC) for example having a thickness of 15 mm, and the body of the module may for example be made of T40 titanium, for example with a thickness of 1.5 mm. The electronics/optical module, the upper cover, the body and the bottom may be made of a TA6V titanium alloy with dimensions for the upper cover for example 32 mm thick, for the body for example 11 mm thick and for the bottom for example 25 mm thick.

The electronics/optical module may be contained for example in a leaktight titanium casing. The dimensions of these chambers are suitable for receiving the device of the invention in two parts, for example from 25 to 30 cm high, for example from 12 to 16 cm in diameter for the containment of the electronics/optical module and for example from 12 to 14 cm high, and for example from 13 to 16 cm long and from 10 to 14 cm wide for the hydraulic module for a device defined in order to be inserted therein.

The present invention also relates to the use of the device of the invention for the analysis of sulfates in a liquid as defined above, for example in a seawater, for example nanofiltered seawater.

For example, in the latter case, the liquid (L) represents said nanofiltered seawater. According to the invention, this nanofiltered seawater may be seawater that is intended to be injected into a natural reservoir or a cavity in order to extract oil, a sampling line (V) possibly enabling this nanofiltered seawater to be sampled in situ in order to thereby analyze the sulfate content thereof.

For example, according to the invention, irrespective of the liquid, said use may comprise the following steps:
a) continuous reversed flow injection, into said injection loop, of a solution for detecting sulfates in said liquid, said solution comprising a stoichiometric amount of BaCl₂ or of a component that makes it possible to detect a maximum concentration of sulfates that is acceptable in said liquid by formation of BaSO₄, or of a sulfate of this other component, it being possible for said detecting solution to additionally comprise thymol crystals and gelatin;
b) injection of said liquid (L) to be analyzed into said injection loop, said liquid to be analyzed pushing said solution injected in step a) and mixing therewith in the mixing loop; and
c) turbidimetric analysis measurement of the BaSO₄, or of the sulfate of the other component, formed where appropriate during step b) via the turbidimetric analysis means (CD).

Advantageously, step a) may be preceded by a step of purging the circuit by means of the liquid (L) by making it circulate, owing to the peristaltic pump (PPA), into the injection and mixing loops and into the turbidimetric analysis means. This purging step advantageously makes it possible to eliminate any residue of a previous analysis and to place the device under optimal conditions for the new analysis.

Advantageously, the measurement step c) may be followed by a step of rinsing the analytical circuit by injection thereinto of a rinsing solution that eliminates the BaSO₄ formed where appropriate, or the sulfate of the other component, it being possible for said rinsing solution to comprise for example ethylenediaminetetraacetate (EDTA) or an equivalent product known to a person skilled in the art that makes it possible to eliminate the BaSO₄ still present in the analytical circuit. Indeed, when BaCl₂ is used to detect the sulfates, in the presence of barium (Ba²⁺), the latter react in order to form a precipitate of barium sulfate (BaSO₄). The Ba²⁺ barium ion of the detecting, or reagent, solution reacts with the SO₄²⁻ sulfate ion of the liquid, or sample, in order to form a precipitate of barium sulfate BaSO₄ that can be detected by turbidimetric analysis at 520 nm.

This precipitate may however, over time, clog the analytical circuit, which may lead to a reduction in the accuracy regarding the measurements, a drop in efficiency and a degradation of the analysis equipment. The injection of a rinsing solution, for example of an alkaline EDTA solution, makes it possible to dissolve any precipitate formed and thus prevent the clogging of the circuit.

This rinsing step may be again followed by a purging step as defined above, before carrying out a new analysis.

According to the invention, the detecting solution may be a solution comprising BaCl₂ or a solution comprising another component known to a person skilled in the art that forms a sulfate that can be detected by turbidimetric analysis or, more generally, a sulfate-containing compound that can be detected by means known to a person skilled in the art that make it possible to detect sulfates. For example, it may be a solution of Fe³⁺ ions, for example when a spectrocolorimetric method is used, resulting in the formation of the compound FeSO₄⁺ detected between 320 and 360 nm, or else a solution of barium chromate BaCrO₄, for example when a spectrocolorimetric method is used with use of an ion-exchange resin, resulting in the release of CrO₄²⁻ chromate ions, the absorbance of which is measured at 370 nm.

According to the invention, advantageously the concentration of BaCl₂ or of component known to a person skilled in the art is a stoichiometric concentration that makes it possible to detect a maximum concentration of sulfates that is acceptable in said liquid by formation of BaSO₄, or of a sulfate of this other component. Thus, if the use of the liquid or the installation in which the liquid circulates or the operator requires that the sulfate concentration of the liquid to be analyzed should not exceed 40 mg·L⁻¹ (0.42×10⁻³ mol/L or 0.42 mM), use will preferably be made of a stoichiometric amount of BaCl₂ or similar component that makes it possible, owing to the device of the present invention and to the use thereof, to detect this maximum threshold. Thus, according to the invention, the amount of reagent used is optimized, limiting the ecological impact, and the amount of precipitate formed in the analytical circuit is minimal, limiting the maintenance operations of this circuit. This optimization comes in addition to that obtained owing to the device of the present invention itself, especially owing to the rFIA reversed flow.

Moreover, according to the invention, the combined addition of thymol crystals and gelatin makes it possible to leave the particles in suspension and therefore to prevent deposits on the walls of the tubes and in the measuring cell. The thymol crystals may be added in a proportion of 0.05 to 0.2 g/L of solution. The gelatin may be added in a proportion of 0.5 to 3 g/L.

For example, according to the invention, use may be made of the following cycle: firstly, the liquid (L) is introduced into the analytical circuit entrained by the peristaltic pump (PPA), for a duration sufficient to purge the circuit. This duration may be from 1 to 5 minutes for example or any other duration that the operator considers appropriate. Secondly, the solution for detecting sulfates, for example a solution of BaCl₂, is introduced into the injection loop having a predefined volume propelled by the peristaltic pump (PPC). Lastly, the liquid is put back into circulation by the peristaltic pump (PPA) into the injection loop, said liquid this time pushing the solution for detecting sulfates, for example BaCl₂ solution, present in the injection loop toward the mixing loop where the liquid and the detecting solution mix. By this mixing, if sulfates are present in the liquid, they react for example with BaCl₂ to form a precipitate of BaSO₄, this precipitate passing before the detection cell where it is detected and measured. At the end of the cycle, rinsing can be carried out, as described above, by injecting the rinsing solution by means of the peristaltic pump (PPB). The device is thus again ready for a new analysis cycle. After injection of the reagent for detecting sulfates, it is possible to carry out a Stop Flow. This allows more time for the chemical reaction to take place and thus makes it possible to obtain a better definition of the signal at the output. This option is particularly advantageous in the case of low sulfate concentrations.

According to the invention, before the implementation of the process of the present invention, it is possible to calibrate the device of the invention by means of one or more standard(s) prepared with artificial seawater and selected concentrations of sulfates. Various turbidimetric analysis measurements are carried out using the device of the present invention, for example at 520 nm when BaCl₂ is used for the solution for detecting sulfates. By this means of artificial seawater, the aqueous matrix of the standards is as faithful as possible to seawater while being free of sulfates or having predetermined concentrations thereof.

The measurement range that can be used owing to the process of the present invention and the use thereof is from 0 to 100 mg of sulfates/L with a detection limit of 4 mg/L (0.04×10⁻³ mol/L or 0.04 mM or 40 μM), which is remarkable.

The device of the present invention makes it possible to become perfectly integrated into an existing offshore oil installation, whereas the means from the prior art are very simply not suitable for the requirements of the oil industry.

The device of the present invention additionally makes it possible, due to its structure, to carry out automated in situ chemical analyses, and this within wide temperature and pressure ranges, for example between 4 and 35° C., and for example between 1×10⁵ and 600×10⁵ Pa (for the deep sea version). For the coastal version, the maximum pressure withstood is 2×10⁵ Pa in operation. And also an in situ calibration of the analyzer by means of “embedded” standards.

This device of the present invention additionally makes it possible to obtain concentration values in real time and thus to rapidly detect anomalies in a device, for example for treating liquid effluents, giving rise to an excessively high content of sulfates in a liquid, for example for injection into reservoirs of natural deposits, for example oil deposits, or any other industrial process, for example those mentioned above. It enables an on-line measurement of the sulfates in a matrix as complex as seawater. The device of the present invention and the use thereof do not require the use of ion-exchange columns before analysis and the calibration of the device may be carried out with standards prepared from artificial seawater.

The device of the present invention also enables a low consumption of energy and of chemical reagents. The amount of reagents used for example in an offshore oil installation is, owing to the present invention, more limited, especially owing to the rFIA reversed flow. This minimal consumption of reagents advantageously makes it possible to limit the maintenance operations on site and to provide long-term autonomy.

The device of the present invention also has the advantage of not requiring personnel on site regularly. The analyzer may indeed be programmed, by means of a programmer, over long periods with an analysis frequency defined by the users. The only maintenance concerns the replacement of the reagents which may be carried out at the end of several months, depending in particular on the volume of reagent reservoir provided for the solution for detecting sulfates.

Other advantages will also appear to a person skilled in the art on reading the examples below, illustrated by the appended figures, given by way of nonlimiting illustration.

### BRIEF DESCRIPTION OF THE FIGURES

FIG. 1 schematically represents a device according to the invention.

FIG. 2 represents a calibration curve for turbidimetric analysis at 420 nm, established from standards of concentrations of sulfates in artificial seawater. Represented on the abscissa is the concentration (C) of sulfates (mg·L⁻¹) and represented on the ordinate is the intensity (I) measured.

FIG. 3 represents a calibration curve for turbidimetric analysis at 810 nm, established from standards of concentrations of sulfates in artificial seawater. Represented on the abscissa is the concentration (C) of sulfates (mg·L⁻¹) and represented on the ordinate is the intensity (I) measured.

FIG. 4a represents calibration curves for turbidimetric analysis at 520 nm (1 GE and 2 GE) established from standards of concentrations of sulfates in artificial seawater, for sulfate concentrations between 0 and 60 mg/L.
Represented on the abscissa of the curve 1 GE is the index of the measurements (an absorbance measurement is carried out every 250 ms) and represented on the ordinate is the absorbance (A) measured (in thousands).
Represented on the abscissa of the curve 2 GE is the concentration (C) of sulfates (mg·L⁻¹) and represented on the ordinate is the intensity (I) measured. The curve 2 GE has the following equation: y=7663.4x+125185 (R²=1)

FIG. 4b represents calibration curves for turbidimetric analysis at 520 nm (1 GE and 2 GE) established from standards of concentrations of sulfates in artificial seawater, for sulfate concentrations between 0 and 60 mg/L.
Represented on the abscissa of the curve 1 GE is the index of the measurements (an absorbance measurement is carried out every 250 ms) and represented on the ordinate is the absorbance (A) measured (in thousands).
Represented on the abscissa of the curve 2 GE is the concentration (C) of sulfates (mg·L⁻¹) and represented on the ordinate is the intensity (I) measured. The curve 2 GE has the following equation: y=6999.9x+120434 (R²=0.9971).

FIG. 5a is a photograph of a contained, coastal version device according to the invention.

FIG. 5b is an exploded schematic representation of the device represented in FIG. 5a.

FIG. 6 is a screenshot of a human-machine interface (HMI) used in one exemplary embodiment of the present invention.

FIG. 7a is a photograph of a contained, deep sea version device according to the invention.

FIG. 7b schematically represents the hydraulic module of the deep sea version device represented in the photograph from FIG. 7a.

FIG. 7c and FIG. 7d schematically represent a hydraulic module of a deep sea version device. Represented schematically in FIG. 7c are bladders of equal pressure (VEQ).

FIG. 7e schematically represents an electronics/optical module of a deep sea version device.

FIG. 8 schematically represents the connection between the detection cell and the hydraulic circuit.

### EXAMPLES

### Example 1

### Analysis Device According to the Invention

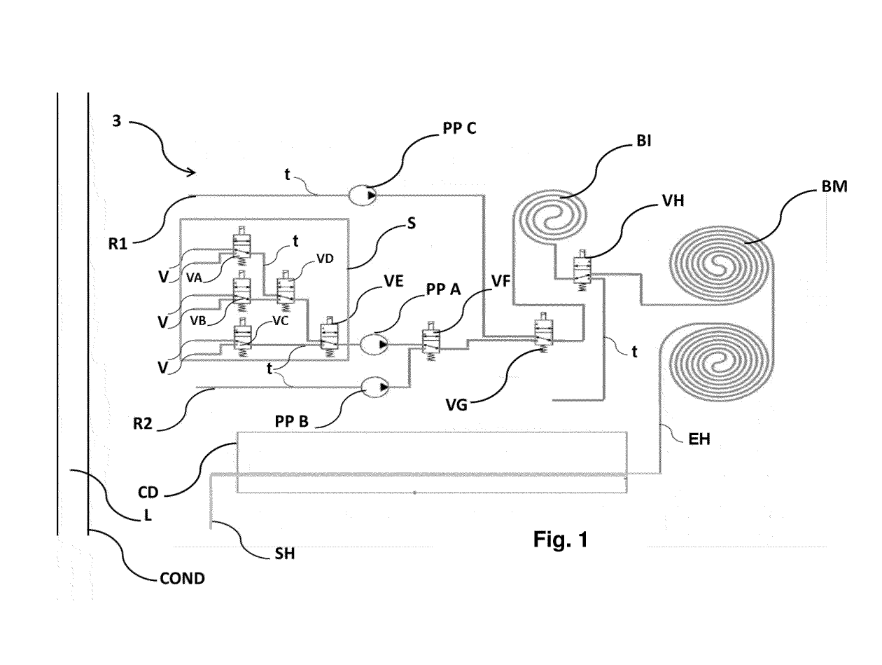

An example of an analysis device according to the present invention is represented schematically in FIG. 1.

The device comprises a leaktight analytical circuit (3) comprising a double injection loop (BI) of defined volume connected to at least one mixing loop (BM), and a turbidimetric analysis means (CD), the injection loop, the mixing loop and the analysis means may be passed through continuously by a liquid, the injection loop and the mixing loop being separated by a valve (VH).

The injection loop is a Teflon (registered trademark) tube of wound-up shape, having an internal diameter of 0.80 millimeter and a length of around 6 centimeters. The predefined volume of this injection loop makes it possible to inject a defined amount of solution for detecting sulfates owing to the peristaltic pump (PPC).

The mixing loop is also a Teflon (registered trademark) tube of wound-up shape. It has an internal diameter of 0.80 millimeter and a length of 2 meters.

The injection and mixing loops are obtained by simple winding. The injection and mixing loops are connected to one another and separated by solenoid valves. The connections between tubes and valves are provided by MINSTAC (registered trademark) (Lee Company, USA) connectors and Luer (trademark) (Fisher, Switzerland) connectors.

The turbidimetric analysis measurement means (CD) comprises 3 LEDs, a photodiode, an electronic detection board (CE) and a detection cell (DC) manufactured by HELLMA (Germany). FIG. 8 schematically represents the detection cell used in this example. Seen in this figure is the detection cell which is passed through by the hydraulic circuit, the incoming optical fiber (FOI) and the outgoing optical fiber (FOS). The tubing (EH) is connected to the cell with the aid of connector technology of Minstac (registered trademark) (Lee Company, USA) type, the tubing at the outlet (SH) may for example be connected to a container for receiving the analyzed liquid (not represented in the diagram) having a length of 3 centimeters and a diameter of 1 millimeter, and an EVOSENS (trademark) (EVOSENS, France) electronic detection board (CE). The electronic board for the detection receives three EVOSENS (trademark) (EVOSENS, France) light-emitting diodes (LEDs), and an EVOSENS (trademark) (EVOSENS, France) photodiode. The light emitted by the light-emitting diodes is transferred from the detection cell to the photodiode-type detector by optical fibers (FO) made of polymethyl methacrylate (PMMA) and having an internal diameter equal to 1 millimeter. The Teflon tube of the mixing loop is connected to the detection cell by a MINSTAC (registered trademark) (Lee Company, USA) connector.

The device also comprises an injection means (IL) for injecting said liquid into said injection loop, said injection means comprising a peristaltic pump (PPA), a system of valves (VF, VG) placed between the pump (PPA) and said injection loop, and a means for sampling said liquid from a source (S).

It also comprises an injection means (ISR) for injecting, into said injection loop, a solution for detecting sulfates in said liquid by turbidimetric analysis; said injection means (ISR) comprising a peristaltic pump (PPC) for injecting said solution for detecting sulfates and a valve (VG) is placed between said pump (PPC) and said injection loop.

The device also comprises an injection means (ISP) for injecting a solution for rinsing said analytical circuit, said injection means (ISP) comprising a peristaltic pump (PPB) for injecting said rinsing solution and a system of valves (VF, VG) placed between the pump (PPB) and said injection loop.

Thus, the analytical circuit of the device includes eight solenoid valves (VA to VH) and three single-channel peristaltic pumps (PPA to PPC) connected together by Tygon R3603 (trademark) (Fisher Scientific, USA) tubing (t) with an internal diameter of 1.14 mm.

The peristaltic pumps (PP) used are Medorex (trademark) (Medorex, Germany) single-channel pumps (OEM Peristaltic-Pump HP/90 42-1-6-1.02×0.8 12 VDC 6VA, 1-channel, 6-rollers). The valves (VN) used are LVM 105R-6 (trademark) (SMC, Japan) marine-grade three-way solenoid valves (one common line, one normally closed line and one normally open line).

These pumps and valves are used for the circulation of the various fluids in the device: reagents, standards and liquid to be analyzed (L). Three of the eight solenoid valves (VF to VH) are used for injecting the reagent into the flow of liquid (L) to be analyzed. The other five valves (VA to VE) are used for the selection of the standards (V lines) or of the liquid to be analyzed (L).

The source (S) comprises six lines (V) for sampling the moving liquid to be analyzed, said lines comprising valves (VA, VB, VC, VD and VE) placed between said moving liquid and the peristaltic pump (PPA). The valves (VA, VB and VC) enable the connection to one or more ducts (COND) in which the liquid to be analyzed circulates.

Transfer pouches (R1, R2) are provided in order to contain the chemical reagents and also the standards necessary for calibrating the analyzer. These transfer pouches are connected to the apparatus via Luer (trademark) (Fisher, Switzerland) connectors. The transfer pouches used for the standards and also for the rinsing solution are made of polyethylene (PE), and have a volume of 600 mL or 1 L. For the reagent solution, the use of a glass container is acceptable.

The solenoid valve (VH) is connected to tubing (t) for discharging liquids circulating in the device, useful for example for the rinsing and purging of the device. This tubing may be connected to a container for receiving the analyzed liquid (not represented).

The turbidimetric analysis measurement means is itself also connected to tubing (t) for discharging liquids circulating in the device, useful for example for the rinsing and purging of the device up to the analysis means. This tubing may be connected to a container for receiving the analyzed liquid (not represented).

The valves, peristaltic pumps and the detection means are connected to electronic means that make it possible to operate the device and control it. This electronic part (not represented) of the device is composed of a board laid out and designed by the inventors on the basis of an ATMEL Atmega (trademark, USA) microcontroller. This board has enabled the management of each means in detail: the speed and the direction of the pumps, the optional supplying of the valves (on/off), the adjustment of the power of the LEDs, the measurement of the power on return, the storage of the measurement data, a communication with the user.

The whole of the device operates under 12 V.

Software was developed by the inventors in Visual Basic; it offers a human-machine interface (HMI) that enables an operator to remotely control the device of the invention. This software makes it possible, through the graphical interface, to actuate the various components of the apparatus (pump, valve, LED, etc.), to program and record cycles (i.e. a script of commands making it possible to automate the sequence of the unit operations necessary for the measurement of a standard or of a sample for example), to export the measurements recorded to calculation software of Excel type.

The device of the invention communicates with the HMI software via an RS-232 serial link. The software can be executed on three levels: a low-level mode, a remote-control mode and an autonomous endurance mode.
In low-level mode, each means and each parameter is accessible manually. For example, in order to modify the rotational speed or else to optionally switch a valve. This mode is useful for primary development and maintenance operations.
In the remote-control mode of the analyzer, a pre-recorded program may be launched. This mode is useful, for example, for acquisitions of a measurement over the short term or during a relatively simple access to the analyzer.
In the autonomous endurance mode, a selection of pre-recorded programs, also referred to as cycles, may be launched. This function is useful for acquiring long series of measurements or for autonomous operations at a set time over several days for example. The data may be stored in an internal memory of the device of the present invention and may be extracted using the operating software of the analyzer (HMI). The raw data may also be processed by the software in order to obtain the value of the light intensity absorbed by the particles (turbidimetric analysis) and to minimize the effect of the noise of the measurements. The raw and processed data may then be transferred from the HMI software to EXCEL calculation software in order to process the data in greater depth.

Moreover, three parameters having an influence on the optical signal can be adjusted by software developed by the inventors: a “Cony” parameter corresponding to the frequency of the sampler, in other words the modulation frequency of the emitting LEDs (“luminescent electronic device”); a “Range” parameter corresponding to the variation of the integration capacitor of the printed circuit enabling synchronous detection; and the “Power” of the LEDs which corresponds to the light intensity of each LED.

### Example 2

### Example of a Coastal Version Device for the Analysis of a Nanofiltered Seawater

The device from example 1 was contained in a leaktight chamber as represented in FIGS. 5a and 5b.

In FIG. 5a, which is a photograph of this coastal version device, it is observed that it is composed of a single block, with, in its upper part, the means for connecting the device to the source of nanofiltered seawater for the analysis thereof.

Seen in FIG. 5b, which is a schematic representation of the coastal device, is the connector (CAI) that enables current to be supplied to the apparatus, and also the communication between the apparatus and the software.

The hydraulic module, namely the injection loop and mixing loop according to the invention, and the electronics/optical module, namely a detection cell (DC), 3 micro-LEDs, a photodiode and an electronic detection board are contained within a leaktight chamber for underwater immersion having a height of 210 millimeters, a length of 148 millimeters and a width of 120 millimeters (FIGS. 5a and 5b).

This chamber is made of PMMA (polymethyl methacrylate) with a thickness of 10 mm. The upper cover is 25 mm thick and the lower cover is 15 mm thick. It was obtained by adhesively bonding the four sides.

FIG. 5b schematically represents the coastal version device without the leaktight chamber.

The connection means between the device and the source of nanofiltered seawater are provided by means of rigid tubing.

The leaktightness at the passage of these connection means through the upper part of the chamber is ensured by PB701 (trademark, Le Joint Français) or 70 Shore A nitrile O-rings.

This device was able to be immersed and was functional down to a depth of 10 meters.

### Example 3

### Deep Sea Version Device for the Analysis of a Nanofiltered Seawater

The deep sea version device used according to example 3 was manufactured in accordance with the coastal version from example 2 by containing the hydraulic module and the electronics/optical module in two separate and leaktight chambers (FIG. 7a).

The leaktightness of the two modules was ensured by placing the hydraulic module at equal pressure in dielectric oil (Fluorinert (trademark) (3M, USA), FC77, 3M, density 1.78), whilst the electronics/optical module was positioned in a leaktight casing made of titanium.

A duct linked to the hydraulic module via a connector (COE) and linked to the electronics/optical module via a connector (CMH) enables the hydraulic module to be supplied with power and enables the command actions of the hydraulic module (FIG. 8).

The electronics/optical module has a connector (CAI) which enables a remote communication between the software and the device and which enables the device to be supplied with power.

The chamber of the electronics/optical module comprises an electronic power supply board (CA) and an electronic measurement board (CEM) (FIG. 7e).

Said device can be immersed down to 6000 meters, and was used in operation down to 2200 meters.

The dimensions of the device being:
for the hydraulic block (MH) (FIGS. 7b and 7c): height 130 mm, length 148 mm, width 120 mm,
for the detection block (MOE) (FIG. 7e): height 264 mm, diameter 140 mm.

### Example 4

### Example of Use of the Device of the Present Invention for the In Situ Analysis of Sulfates in a Nanofiltered Seawater

The experiments were carried out with the device described in example 2.

The source (S) of the device is connected to a seawater nanofiltration module via said lines (V), the nanofiltered seawater constituting the liquid to be analyzed.

### Reagents and Solutions Used:

### Example of Solution for Detecting Sulfates

The solution for detecting sulfates selected is a solution of barium chloride (BaCl₂). By stirring using a magnetic stirrer bar, 0.20 g of thymol (5-methyl-2-(propan-2-yl)phenol) crystals (Sigma-Aldrich, registered trademark, USA) were dissolved in 500 ml of hydrochloric acid (Sigma-Aldrich, registered trademark, USA) having a concentration of 0.005 mol·L⁻¹ at a temperature of around 80° C. The solution was then cooled to around 40° C. and 1500 ml of hydrochloric acid having a concentration of 0.005 mol·L⁻¹ were added to the solution. 4 g of gelatin (Sigma-Aldrich, registered trademark, USA) were added carefully, then the mixture was stirred until the gelatin had dissolved. Finally, 20 g of dehydrated barium chloride (Sigma-Aldrich, registered trademark, USA) were dissolved. The mixture was filtered and placed in transfer pouches.

### Example of Rinsing Solution

The rinsing solution prepared is an alkaline solution of EDTA. For this, 40 g of EDTA (Fluka Analytical, registered trademark, USA), 7 g of ammonium chloride (Sigma-Aldrich, registered trademark, USA) and 120 ml of a concentrated ammonia solution (Fluka Analytical, registered trademark, USA) were dissolved in 500 ml of Milli-Q water (resistivity of 18.2 MO, temperature of 25° C.) (pH=10). The mixture obtained was then diluted to 1 liter with demineralized water.

### Example of Standard Solutions of Sulfates

A stock solution of sulfates (5000 mg·L⁻¹) was prepared by dissolving 9.05 g of potassium sulfate K₂SO₄ (Sigma-Aldrich, registered trademark, USA) in one liter of demineralized water. The standard solutions (0, 10, 20, 30, 40, 50, 60, 70, 80, 90 and 100 mg·L⁻¹) were prepared by diluting an appropriate volume of stock solution in demineralized water or in a sulfate-free synthetic seawater solution or sulfate-free artificial seawater solution.

### Example of Test Solution: Sulfate-Free Synthetic Seawater Solution

Sulfate-free synthetic seawater was prepared by diluting the following products in 1900 ml of demineralized water:

Anhydrous salts:
49.06 g of NaCl
0.2 g of KBr
0.4 g of NaHCO₃
1.4 g of KCl
0.06 g of H₃BO₃
0.06 g of NaF

Hydrated salts:
22.2 MgCl₂.6 H₂O
3.08 g of CaCl₂.2 H₂O
0.34 g of SrCl₂.6 H₂O

The mixture obtained was then topped up to 2 liters with demineralized water.

### Use of the Device of the Invention for the Analysis of Sulfates in a Liquid

Firstly, the liquid to be analyzed was introduced into the device of the present invention as described in example 1, in the injection loop, then was entrained by a peristaltic pump A (PPA) for 80 seconds corresponding to the purging of the analytical circuit.

Secondly, the solution (R1) comprising a stoichiometric amount of BaCl₂ was introduced into the injection loop (BI) which has a predefined volume. The reagent was then propelled by the peristaltic pump C (PPC). Finally, the sample was put back into circulation by the peristaltic pump A, but this time pushed the solution comprising a stoichiometric amount of BaCl₂ contained in the injection loop.

After reaction between the liquid to be analyzed and the BaCl₂ in the mixing loop, the precipitate passed before the turbidimetric analysis means (CD) where it was analyzed before being discharged into a container for receiving the analyzed liquid (not represented). The formation of the precipitate was then detected toward 420 nm for the range 0 to 40 mg. L⁻¹ or 810 nm for the range 40 to 100 mg·L⁻¹.

At the end of the cycle, a rinsing of the circuit was carried out using an alkaline solution of ethylenediaminetetraacetate (EDTA) (R2) propelled by the peristaltic pump B.

When the liquid analyzed contained low concentrations of sulfates (between 0 and 60 mg·L⁻¹), the flow was stopped for 9 minutes when said liquid to be analyzed and the solution comprising BaCl₂ were in the mixing loop in order to detect lower concentrations of sulfates and in order to obtain a more sensitive signal.

The addition of an alkaline solution of EDTA made it possible to dissolve the precipitate formed and thus made it possible to prevent the clogging of the circuit. The combined addition of thymol crystals and gelatin made it possible to leave the particles in suspension and therefore prevent the deposits on the walls of the tubes and in the detection cell.

The measurement range and the accuracy associated with this study range were able to be modified by varying the wavelength of the LEDs.

All the reagents were prepared with Milli-Q water (Millipore (trademark)).

The analysis device was programmed and controlled by human-machine interface operating software of the analyzer developed in Visual Basic and which was able to be executed on three levels: the low-level mode, the remote-control mode and the autonomous endurance mode.
In low-level mode, each means and each parameter is accessible manually. For example, in order to modify the rotational speed or else to optionally switch a valve. This mode is useful for primary development and maintenance operations.
In the remote-control mode of the analyzer, a pre-recorded program may be launched. This mode is useful, for example, for acquisitions of a measurement over the short term or during a relatively simple access to the analyzer.
In the autonomous endurance mode, a selection of pre-recorded programs, also referred to as cycles, may be launched. This function is useful for acquiring long series of measurements or for autonomous operations at a set time over several days for example. The data may be stored in an internal memory of the device of the present invention and may be extracted using the operating software of the analyzer (HMI). The raw data may also be processed by the software in order to obtain the value of the light intensity absorbed by the particles (turbidimetric analysis) and to minimize the effect of the noise of the measurements. The raw and processed data may then be transferred from the HMI software to EXCEL calculation software in order to process the data in greater depth.

### Development of the Calibration Curves:

Calibration Curves for Sulfate Concentrations Between 0 and 100 mg/L in the Standard Sulfate Solutions

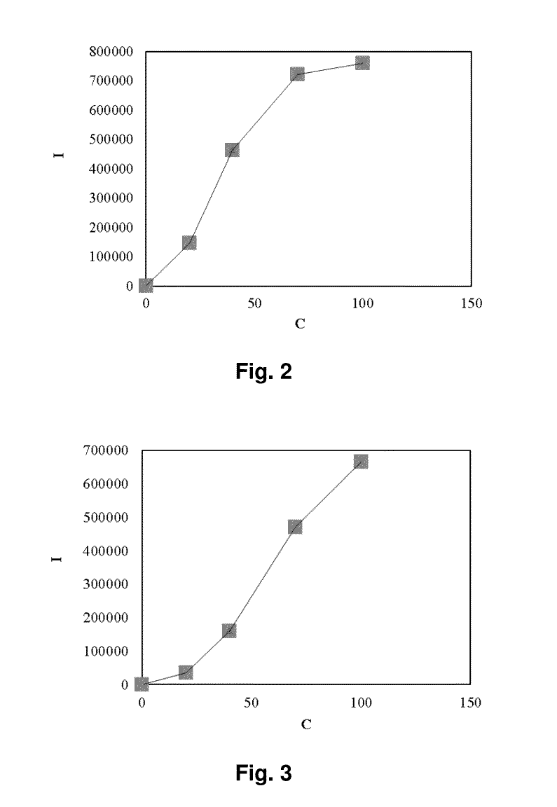

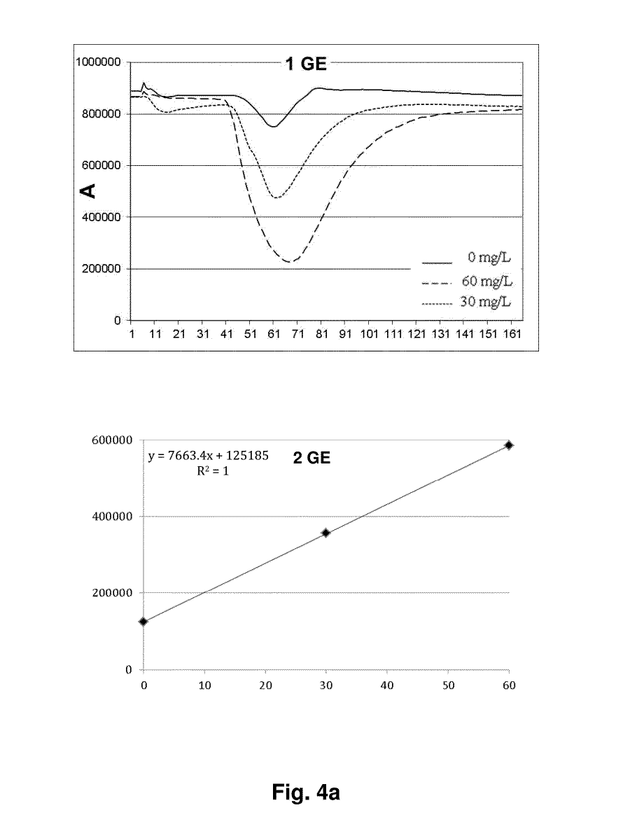

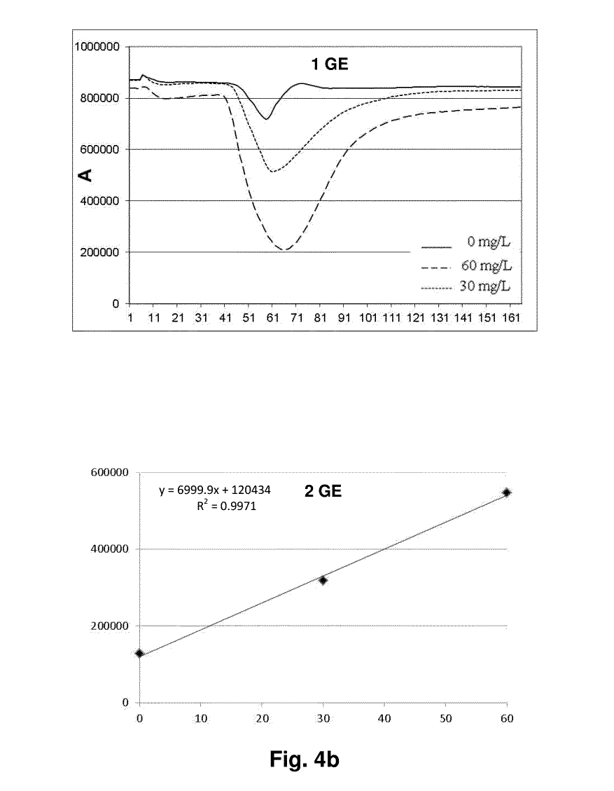

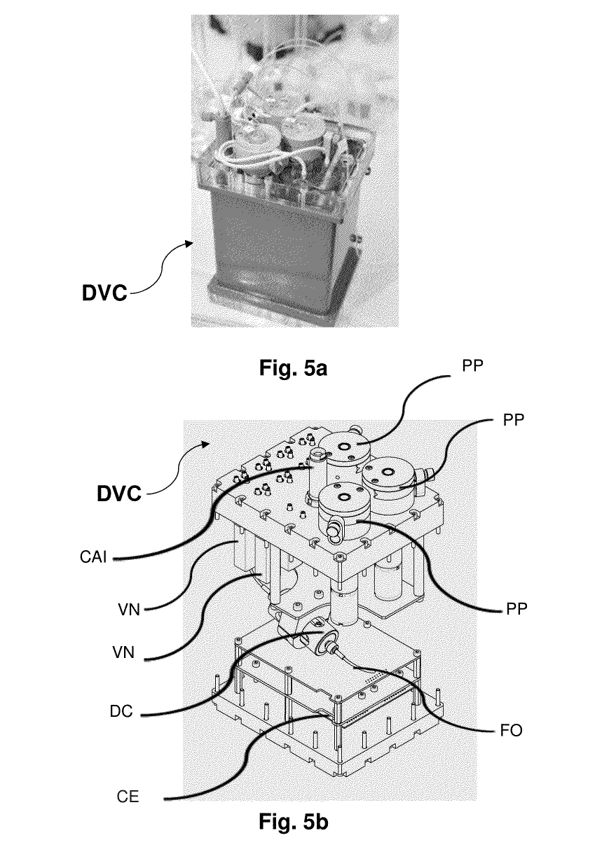

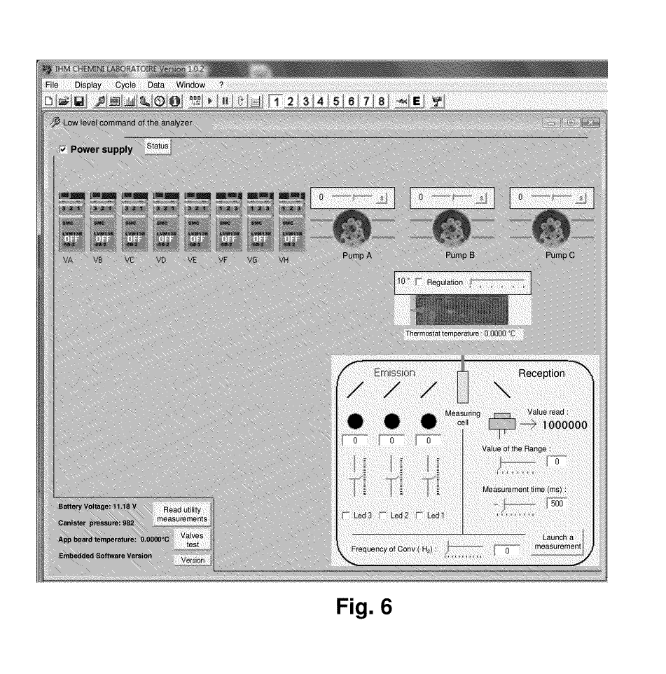

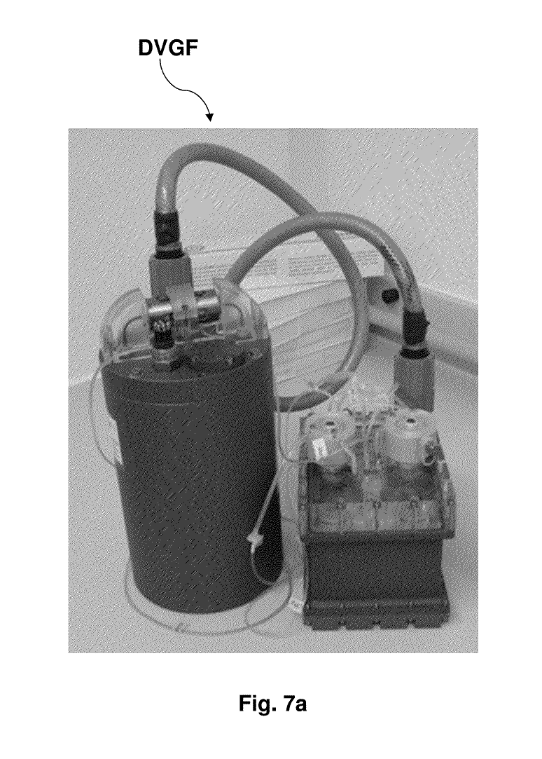

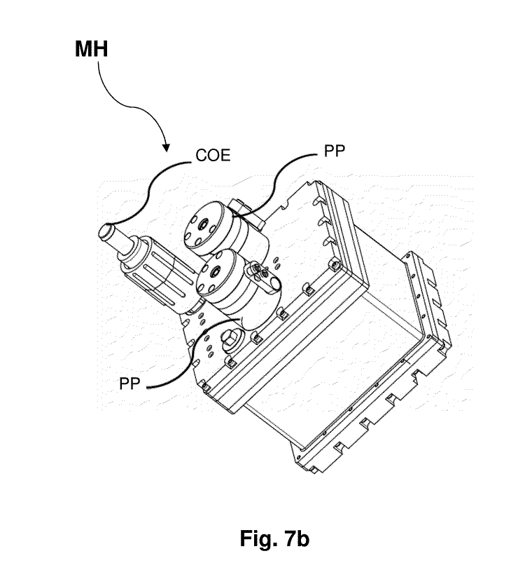

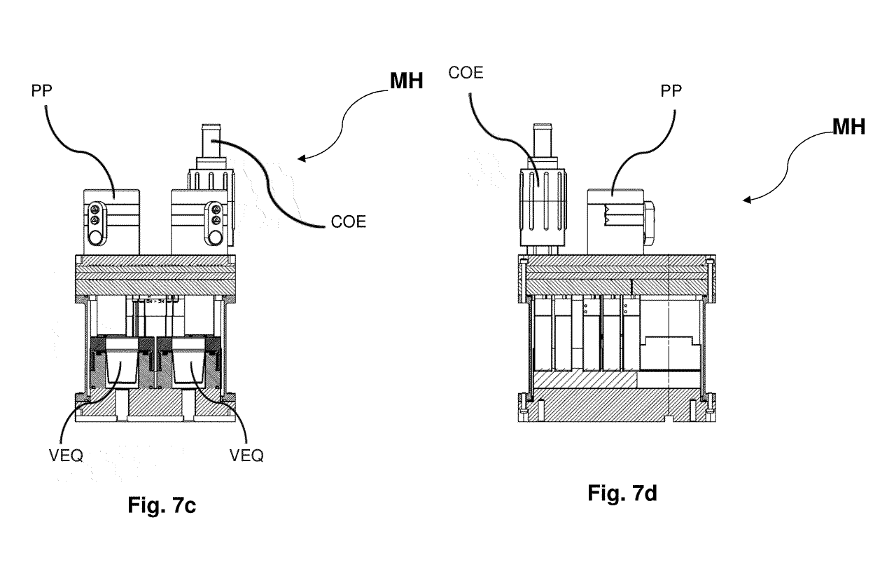

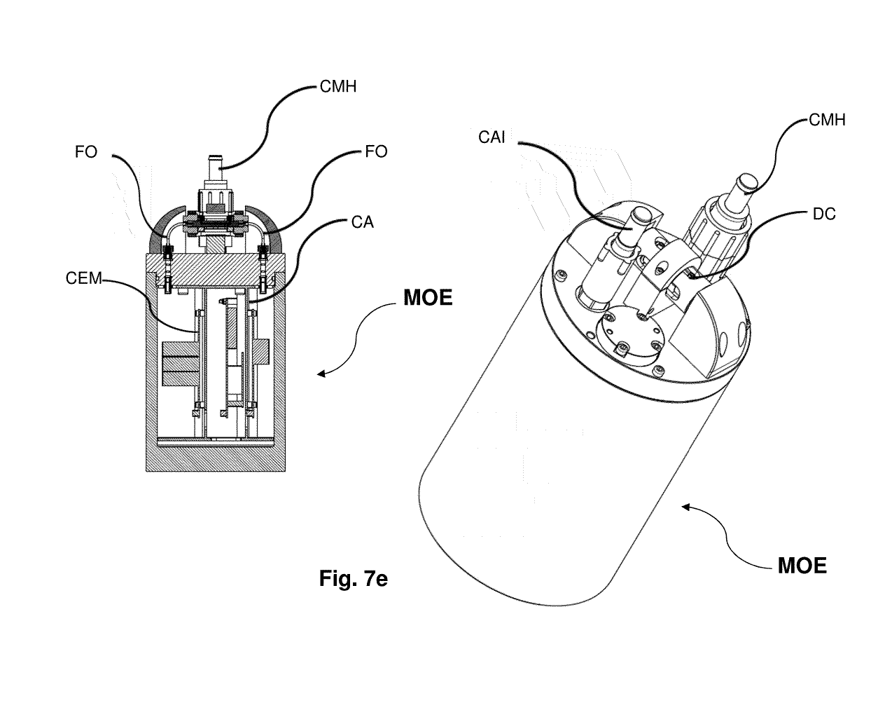

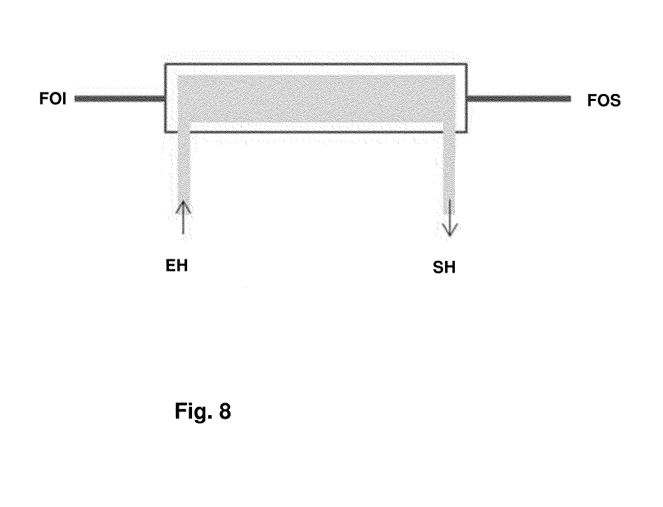

Calibration curves were established according to the experimental protocol described above, from standards having sulfate concentrations of 0, 20, 40, 70 and 100 mg·L⁻¹ prepared as above, for wavelengths of 420 nm (FIG. 2) and 810 nm (FIG. 3).

Calibration Curves for Sulfate Concentrations Between 0 and 60 mg/L

Calibration curves were also produced for values of sulfate concentrations between 0 and 60 mg·L⁻¹. The analysis of a range of sulfate standards (0, 30 and 60 mg·L⁻¹) by the device as described above gave the results which are represented in FIGS. 4a and 4b. These curves were obtained with the use of a Stop Flow of 9 minutes after injection of the reagent BaCl₂. This stop allows the reaction more time to develop and thus makes possible an increased sensitivity.

### Nanofiltered Seawater:

A nanofiltered seawater with a salinity of 24.3%0 was obtained after nanofiltration of a seawater originating from the Mediterranean Sea by the Total pilot plant installed at the Ifremer station in Palavas-Les-Flots.

Comparison of the Results Obtained with Those Obtained by Ion Chromatography:

A measurement series was carried out in the laboratory to obtain concentrations over four days of handling on a nanofiltered seawater resulting from the Palavas-Les-Flots nanofiltration pilot plant. The objective being to compare the concentration values obtained by the turbidimetric analysis method by continuous reversed flow injection according to the invention with those obtained by ion chromatography carried out by the Ifremer Brest Environnement Profond (Deep Environment) laboratory over the four days of handling.

Comparison of the Results Obtained Over Several Days with Those Obtained by Ion Chromatography:

Values obtained by ion chromatography measurement
The samples were prepared by acidification to 1%0 by 65% nitric acid then dilution by 3 and by 5. The samples were stored at ambient temperature.

Two samples of nanofiltered seawater with a salinity of 24.3%0 were sampled and analyzed. The average concentration after calculation with the dilution coefficient is 90.1 mg·L⁻¹ with a standard deviation of 1.5 mg·L⁻¹.
Values obtained by the turbidimetric analysis method by continuous reversed flow injection according to the invention

The measurements of the amounts of sulfates contained in the liquids analyzed were carried out in accordance with the experimental protocol described above. The results are listed in table 1 below:

TABLE 1
Average concentrations for each day of the handling. The
standard deviations are indicated between parentheses.

| Day | Day 1 | Day 2 | Day 3 | Day 4 |
|---|---|---|---|---|
| Average concentration | 89.0 | 89.9 | 88.2 | 90.1 |
| (mg · L⁻¹) | (5.5) | (3.0) | (3.0) | (3.5) |

Firstly, the results obtained show that the measured concentration values of the sample are similar since they vary between 88.2 and 90.1 mg·L⁻¹. Furthermore, these values are also similar to the value determined by ion chromatography which was 90.1 mg·L⁻¹.

Consequently, the method has analytical performances in agreement with the requested specifications.

### Analytical Performances of the Analyzer

The analytical performances of the analyzer were evaluated according to the experimental protocol described above. The results are listed in table 2 below:

TABLE 2
Analytical performances of the analyzer according to the invention.

| | | | Accuracy |
|---|---|---|---|
| | | | (at 10 mg · L⁻¹) |
| Range | Detection limit | Quantification limit | (at 40 mg · L⁻¹) |
| 0-60 mg · L⁻¹ | 4 mg · L⁻¹ | 10 mg · L⁻¹ | 5%-1.1% |

The results show that the quantification limit and the detection limit are in agreement with the criterion of sulfate concentrations tolerated in the use of nanofiltered seawater in the exploitation of deposits, the threshold value being around 40 mg·L⁻¹.

The accuracy (or “coefficient of variation” defined as the ratio between standard deviation and the average multiplied by 100) obtained for a standard of 10 mg·L⁻¹ is 5% (acceptable for an in situ analyzer) and it is 1.1% for a standard at 40 mg·L⁻¹ (target value). These parameters were obtained by working in the range 0 to 60 mg·L⁻¹.

1. A device for analyzing sulfates in a liquid, said device comprising:
a) a leaktight analytical circuit comprising an injection loop of defined volume connected to at least one mixing loop, and a turbidimetric analysis means, it being possible for the injection loop, the mixing loop and the analysis means to be passed through continuously and successively by the liquid, the injection loop and the mixing loop being separable by a valve;
b) an injection means for injecting said liquid into said injection loop, said injection means comprising a peristaltic pump, a system of valves placed between the pump and said injection loop, and a means for sampling said liquid from a source;
c) an injection means for injecting, into said injection loop, a solution for detecting sulfates in said liquid by turbidimetric analysis; said injection means comprising a peristaltic pump for injecting said solution for detecting sulfates and a valve placed between said pump and said injection loop; and
d) an injection means for injecting a solution for rinsing said analytical circuit, said injection means comprising a peristaltic pump for injecting said rinsing solution and a system of valves placed between the pump and said injection loop.

2. The device as claimed in claim 1, wherein the source comprises at least one line for sampling the moving liquid, said line comprising at least two valves placed between said moving liquid and the peristaltic pump.

3. The device as claimed in claim 2, wherein said source can be connected to a seawater nanofiltration module via said line, the nanofiltered seawater constituting said moving liquid.

4. The device as claimed in claim 1, said device being automated by means for programming and controlling the continuous reversed flow injection, into said injection loop, of the solution for detecting sulfates in said liquid, said solution comprising a stoichiometric amount of BaCl₂ that makes it possible to detect a maximum concentration of sulfates that is acceptable in said liquid.

5. The device as claimed in claim 1, additionally comprising a memory adapted to memorize successive analyses.

6. The device as claimed in claim 1, additionally comprising Human-Machine Interface operating software developed in Visual Basic.

7. The device as claimed in claim 1, said device being composed of two parts, one comprising the injection and mixing loops and the other comprising the turbidimetric analysis means.

8. The device as claimed in claim 1, said device being contained within at least one leaktight chamber for underwater immersion.

9. The device as claimed in claim 1, wherein the device is configured for the analysis of sulfates in a nanofiltered seawater, said liquid being said nanofiltered seawater.

10. The device as claimed in claim 9, wherein the nanofiltered seawater is that injected into a cavity in order to extract oil, the sampling line enabling this nanofiltered seawater to be sampled in situ in order to thereby analyze the sulfate content thereof.

11. The device as claimed in claim 9, wherein the device is further configured for:
a) continuous reversed flow injection, into said injection loop, of the solution for detecting sulfates in said liquid, said solution comprising a stoichiometric amount of BaCl₂ that makes it possible to detect a maximum concentration of sulfates that is acceptable in said liquid by formation of BaSO₄, it being possible for said reactive solution to additionally comprise thymol crystals and gelatin;
b) injection of said liquid to be analyzed into said injection loop, said liquid to be analyzed pushing said solution injected in step a) and mixing therewith in the mixing loop; and
c) turbidimetric analysis measurement of the BaSO₄ formed where appropriate during step b) via the turbidimetric analysis means.

12. The device as claimed in claim 11, wherein step a) is preceded by a step of purging the circuit by means of the liquid by making it circulate, owing to the peristaltic pump, into the injection and mixing loops and into the turbidimetric analysis means.

13. The device as claimed in claim 11, wherein the measurement step c) is followed by a step of rinsing the analytical circuit by injection thereinto of a rinsing solution that eliminates the BaSO₄ formed where appropriate, it being possible for said rinsing solution to comprise EDTA.

14. The device as claimed in claim 11, wherein a calibration of the device is carried out by means of one or more standard(s) prepared with artificial seawater.

15. The device as claimed in claim 1, further configured for analysis of sulfate in a liquid intended for or resulting from an industrial process.
